SEQUENCE LISTING (16 sequences)

SEQ ID NO: 1 - DNA, 338 bp, Homo sapiens
gagtttaacctgacaatttcttcttgttctctattcttttgattgagaagctcctgtcgccgaattctctcccattctaagcgacgttgtcgttcaagttcctgttttgctgcctctcgtctttctatgtcttttctcctttcttcctctcgttgtctctccaattcccgttgcttctctaagcgtttttctaattcaagttgtttcttccattcttgttcttgtaattctctctgttttcgttcccactcttccttttctttctgggctttacgttctgcctccctttgttgctgctccatcaaggcttggcgtcgcttttccagctccatgttccc

SEQ ID NO: 2 - DNA, 17 bp, Artificial Sequence
  Synthetic
gttttttttttttttta

SEQ ID NO: 3 - DNA, 17 bp, Artificial Sequence
  Synthetic
gtttttttttttttttc

SEQ ID NO: 4 - DNA, 17 bp, Artificial Sequence
  Synthetic
gtttttttttttttttg

SEQ ID NO: 5 - DNA, 10 bp, Artificial Sequence
  Synthetic
ctttgagcga

SEQ ID NO: 6 - DNA, 20 bp, Artificial Sequence
  Synthetic
gccaactatgagcgagggaa

SEQ ID NO: 7 - DNA, 20 bp, Artificial Sequence
  Synthetic
tgttgctgctccatcaaggc

SEQ ID NO: 8 - DNA, 22 bp, Artificial Sequence
  Synthetic
tggagctggaaaagcgacgcca

SEQ ID NO: 9 - DNA, 25 bp, Artificial Sequence
  Synthetic
tcacccacactgtgcccatctacga

SEQ ID NO: 10 - DNA, 25 bp, Artificial Sequence
  Synthetic
cagcggaaccgctcattgccaatgg

SEQ ID NO: 11 - DNA, 26 bp, Artificial Sequence
  Synthetic
atgccctcccccatgccatcctgcgt

SEQ ID NO: 12 - DNA, 26 bp, Artificial Sequence
  Synthetic
accagcaagagttctctatagctatg

SEQ ID NO: 13 - DNA, 26 bp, Artificial Sequence
  Synthetic
ctgtaagatgatgcatgaggcaatgt

SEQ ID NO: 14 - DNA, 27 bp, Artificial Sequence
  Synthetic
tcatcagccattgcctccagttgcacc

SEQ ID NO: 15 - DNA, 5828 bp, Artificial Sequence
  Synthetic
cgccagatggctgagagctatcaaggaaaactcaggaccatgatggctcagtttcccacagctatgaatggagggccaaacatgtgggctattacctctgaagaacgtactaagcatgacaggcagtttgataacctcaaaccttcaggaggttacataacaggtgatcaagcacgtaattttttcctacaatcaggtctgccggcccctgttttagctgaaatatgggctttatcagacctaaacaaggatgggaagatggatcagcaagagttctccatagctatgaaactcatcaaactgaagcttcaaggccaacagttgcctgtggttctccctcctattatgaagcaaccccctatgttttctccattaatttctgctcgttttggaatgggaagcatgcccaatctgtccattcctcagccattgcctccagctgcacctataacatcattgtcttctgcgacttcagggaccaaccttcctcccttaatgatgcccactcccctagtgccttctgttagcacatcatcattaccaaatggaaccgccagtctcattcagcctttacccattccttattcttcttcaacattgcctcatgggtcatcttatagtctgatgatgggaggatttggaggtgctagtatacagaaagcgcagtctctgattgatttaggatctagtagctcaacttcctcgactgcttcactctcagggaactcacccaagactgggacctcagagtgggcagttcctcagcctacaagattaaaatatcggcaaaaatttaatactcttgacaaaagtatgagtggatatctctcaggttttcaagctagaaatgcccttcttcagtcaaatctttctcaaactcagctggctactatttggactctggctgacgttgatggtgatggacagctaaaagcagaagagtttattcttgcaatgcaccttactgacatggccaaagctggacagccattaccactgactttacctcctgagcttgttcctccatctttcagaggaggaaagcaaattgattccattaatggaactctgccttcatatcagaaaatgcaagaagaggagcctcagaagaaattaccagttacttttgaggacaaacggaaagccaactatgagcgagggaacatggagctggaaaagcgacgccaagccttgatggagcagcaacaaagggaggcagaacgtaaagcccagaaagaaaaggaagagtgggaacgaaaacagagagaattacaagaacaagaatggaagaaacaacttgaattagaaaaacgcttagagaagcaacgggaattggagagacaacgagaggaagaaaggagaaaagacatagaaagacgagaggcagcaaaacaggaacttgaacgacaacgtcgcttagaatgggagagaattcggcgacaggagcttctcaatcaaaagaatagagaacaagaagaaattgtcaggttaaactctaaaaagaagaatcttcatcttgagttggaagcactgaatggcaaacatcagcagatctcaggcagacttcaggatgtccgactcaaaaagcaaactcaaaagactgagctggaagttctggataagcagtgtgacttggaaattatggaaatcaagcaacttcaacaggaacttcaggaatatcagaataagcttatctatctggtacctgagaagcaattattaaatgaaagaattaaaaacatgcagttcagtaacacacctgattcaggggtcagtttacttcataaaaaatcattagaaaaggaagaattatgccaaagacttaaagaacagttagatgctcttgaaaaagaaactgcatctaagctgtcagaaatggattcttttaacaatcaactaaaggaactgagagaaacctacaacacacagcagttagcccttgaacagctttataagatcaaacgtgacaagttgaaggaaattgaaaggaaaagattagaactaatgcagaaaaagaaactagaagatgaggctgcaaggaaagcaaagcaaggaaaagaaaacttatggaaagaaaatcttagaaaggaggaagaagaaaaacaaaagcgactccaggaagaaaaaacacaagaaaaaattcaagaagaggaacggaaagctgaggagaaacaacgtaaggataaggatactttgaaagctgaggagaaaaaacgtgagacagctagtgttttggtgaattatagagcattatacccctttgaagcaaggaaccatgatgagatgagttttaattctggagatataattcaggttgatgaaaaaaccgtaggagaacctggttggctttatggtagttttcaaggaaattttggctggtttccatgcaattatgtagaaaaaatgccatcaagtgaaaatgaaaaagctgtatctccaaagaaggccttacttcctcctacagtttctttatctgctacctcaacttcctctgaaccactttcttcaaatcaaccagcatcagtgactgattatcaaaatgtatctttttcaaacctaactgtaaatacatcatggcagaaaaaatcagccttcactcgaactgtgtcccctggatctgtatcacctattcatggacagggacaagtggtagaaaacttaaaagcacaggccctttgttcctggactgcaaagaaagataaccacttgaacttctcaaaacatgacattattactgtcttggagcagcaagaaaattggtggtttggggaggtgcatggaggaagaggatggtttcccaaatcttatgtcaagatcattcctgggagtgaagtaaaacgggaagaaccagaagctttgtatgcagctgtaaataagaaacctacctcggcagcctattcagttggagaagaatatattgcactttatccatattcaagtgtggaacctggagatttgactttcacagaaggtgaagaaatattggtgacccagaaagatggagagtggtggacaggaagtattggagatagaagtggaatttttccatcaaactatgtcaaaccaaaggatcaagagagttttgggagtgctagcaagtctggagcatcaaataaaaaacctgagattgctcaggtaacttcagcatatgttgcttctggttctgaacaacttagccttgcaccaggacagttaatattaattctaaagaaaaatacaagtgggtggtggcaaggagagttacaggccagaggaaaaaagcgacagaaaggatggtttcctgccagtcatgttaaacttttgggtccaagtagtgaaagagccacacctgcctttcatcctgtatgtcaggtgattgctatgtatgactatgcagcaaataatgaagatgagctcagtttctccaagggacaactcattaatgttatgaacaaagatgatcctgattggtggcaaggagagatcaacggggtgactggtctctttccttcaaactacgttaagatgacgacagactcagatccaagtcaacagtggtgtgctgatctgcaaaccctggacacaatgcagccaattgagaggaaaagacagggctatattcatgagctgattcagaccgaagagcggtacatggctgaccttcagctcgtcgtcgaggtttttcagaaacgcatggcagagtcaggctttctcactgaaggggagatggccctgatttttgttaactggaaggagctcatcatgtccaacacaaagctgctgaaggctttgcgggtgcggaagaagaccgggggcgagaagatgccggtgcagatgattggggacatcctggccgctgagctgtcccacatgcaggcttacatcaggttctgcagctgccagcttaatggagcagctctgttacagcagaagacagatgaagacacagatttcaaagaatttttaaagaagctggcatctgacccgcggtgtaaaggaatgcccctctccagcttcctgctgaaacccatgcagaggatcacccgctacccactgctcatcagaagtattctggagaacaccccggagagccatgcagaccattcctccctaaagctggccctcgagcgggcagaggagctgtgctctcaagtgaatgagggagttcgggagaaggaaaactcggaccgactggagtggatccaggcgcacgtgcagtgtgaaggcctcgcggagcaacttattttcaactctctcaccaactgcctggggccccggaagctcttacacagtgggaaattatacaagaccaagagcaacaaggaactgcacggattcctcttcaatgacttcctgcttcttacctacatggtcaagcagtttgctgtttcctctggctctgagaaacttttcagctcgaagtccaatgctcaattcaaaatgtataaaacgcccattttcctgaatgaagtcttggtgaaactgcccacagacccttccagcgatgagcctgtcttccacatttcccacattgatcgggtctacaccctccgaacagacaacattaatgagaggaccgcctgggtgcagaagatcaaggcggcgtctgagcagtacatcgacaccgagaagaagcagcgtgagaaagcttaccaagcccgctcccaaaagacttcaggcattgggcgcctgatggtgcatgtcattgaagctacagaattaaaagcctgcaaaccaaatggaaagagcaacccatactgtgaaatcagcatgggctcccagagctacaccaccaggaccatccaggacacactcaatcccaagtggaattttaactgccagttctttattaaggatctctaccaagacgtgctgtgtctcaccctgtttgacagagaccagttttcaccagatgatttcctgggtcgtactgaaattccagtggcaaaaattcgaacagaacaggaaagcaaaggccctatgacccgccgactgctgctgcatgaggtccccaccggggaggtctgggtccgttttgacctgcagctttttgagcaaaaaactctcctgtaggggttctaaaggacagcaccagcgggacagcccacaaggctggggctggagaatgagagactgcgctctcttggggctgagggagcaccatgcagcttcacccctcacaaagccatgcacgctgggggctctgttttcctgcacactaaatagctagcaatctatgcaaacacctttcccataaagaaaccaaaccccatagtacagtgccttgtcctagtgttcacatgttcagctctgtttgtttagatgccaaggtttccattttcagggctataaaaagtattacttggaaatgaggcatcagaccaccagatgttaccgctcggttgaatgtgtccaccgtggagtggtttggtgacgctgtaaccattccacgccagtgacctctgctgggtcacagccactcaggaggggaagggtcaggatgagaggctgcagcctcgacacttggcgcggcctgatactgaaatagcgtctactcgtgcactgaataaaaacagaaacttgatcattttattcctgattagattttatcactctctgctaagacaatatagtctggagtataagtgggaaagcttgatttaaatactgtgaactctaataatgtggaaaatatttttcaactttaattttctgaagtataaattatttatgtaaattcattgtttttgcatatttcttaggacatgcatctttaagctttatcattgcccatatgtacagaaagagaataaagacatatgtttatggatgaaaaaaaaaaaaaaaaa

SEQ ID NO: 16 - protein, 1681 aa, Artificial Sequence [sequence omitted]

(19) United States
(12) Patent Application Publication
(10) Pub. No.: US 2004/0053282 A1
(43) Pub. Date: Mar. 18, 2004

(54) Method of examining allergic disease

(76) Inventors: Yuji Sugita, Ibaraki (JP); Ryoichi Hashida, Ibaraki (JP); Kaoru Ogawa, Ibaraki (JP); Takeshi Nagasu, Ibaraki (JP); Eiki Takahashi, Ibaraki (JP); Hirohisa Saito, Tokyo (JP); Masaya Obayashi, Tokyo (JP)

Correspondence Address: Peter G Carroll, Medlen & Carroll, Suite 350, 101 Howard Street, San Francisco, CA, 94105, US

(21) Appl. No.: 10/398,885

(22) PCT Filed: Oct. 11, 2001
(86) PCT No.: PCT/JP01/08937

(30) Foreign Application Priority Data
Oct. 13, 2000 (JP) 2000-314093

(51) Int. Cl. C12Q 1/68; C12P 19/34

(52) U.S. Cl. 435/6; 435/91.2

(57) ABSTRACT

The differential display method was used to search for a gene whose expression level in eosinophils of patients with atopic dermatitis. As a result, intersectin 2 was isolated as a gene showing a significant increase in expression in eosinophils of light patients. The gene is usable in testing for an allergic disease and screening for a candidate compound for a therapeutic agent therefor an allergic disease.

### TECHNICAL FIELD

The present invention relates to methods of testing for allergic diseases.

### BACKGROUND ART

Allergic diseases, such as atopic dermatitis, are considered to be multifactorial diseases. These diseases are caused by the interaction of many different genes, whose expressions are influenced by various environmental factors. Thus, the determination of specific genes causing a specific disease has been extremely difficult for allergic diseases.

Additionally, expression of mutated or defective genes, or overexpression or reduced expression of specific genes is thought to be involved in allergic diseases. To elucidate the role of gene expression in diseases, it is necessary to understand how a gene is involved in triggering disease onset and how the expression of the gene is altered by external stimulants, such as drugs.

Recent developments in gene expression analysis techniques have enabled analysis and comparison of gene expression in many clinical samples. Among these methods, the differential display (DD) method is useful. The differential display method was originally developed by Liang and Pardee in 1992 (Science, 1992, 257: 967-971). According to this method, several tens or more different samples can be screened at one time to detect genes whose expressions differ among the samples. Important information vis-à-vis causative genes of a disease is expected to be uncovered through examining genes with mutations or genes whose expression changes depending on time and environment. Such genes include those whose expression is influenced by environmental factors.

History taking, and confirmation of family history and anamnesis of the patient are important in general for recent diagnosis of allergic diseases. Further, methods of diagnosing allergy based on more objective information include a method in which patient's blood sample is tested and method of observing patient's immune response to allergen. Examples of the former method include the allergen-specific IgE measurement, leukocyte histamine release test, and lymphocyte stimulating test. The presence of allergen-specific IgE verifies the allergic reaction against the allergen. However, allergen-specific IgE is not always detected in every patient. Furthermore, the IgE assay requires performing tests for all of the allergens necessary for diagnosis. The leukocyte histamine release test and lymphocyte stimulating test are methods for observing the reaction of the immune system toward a specific allergen in vitro. These methods require complicated operation.

Another known method of allergy diagnosis is based on the immune response observed at the time when a patient is contacted with an allergen (example of the latter method). Such tests include the prick test, scratch test, patch test, intradermal reaction, and induction test. These tests allow for the direct diagnosis of patient's allergic reaction, but are regarded as highly invasive tests because patients are actually exposed to allergen.

In addition, regardless of the allergen types, methods to testify the involvement of allergic reaction are also attempted. For example, a high serum IgE titer indicates the occurrence of allergic reaction in a patient. The serum IgE titer corresponds to the total amount of allergen-specific IgE. Though it is easy to determine the total amount of IgE regardless of the type of allergen, the IgE titer may be reduced in some patients, for example, those with non-atopic bronchitis.

The number of eosinophils and eosinophil cationic protein (ECP) value are used to diagnose delayed-type reaction following Type I allergy and allergic inflammatory reaction. The number of eosinophils is considered to reflect the progress of allergic symptoms. ECP, a protein contained in eosinophil granules, is also strongly activated in patients with an asthma attack. Even though these diagnostic items reflect allergy symptoms, in reality, the general observation is that increase of eosinophils becomes clearly noticeable with advancement of allergic symptoms. That is, the period in which an increase in eosinophils is clearly observed is often accompanied by marked allergic symptoms. Therefore, the number of eosinophils cannot be used as an indicator for the early stage of an allergic disease.

Therefore, a marker for an allergic disease that is not dangerous towards patients and that can allow for easy acquisition of information necessary for early diagnosis, would be useful. Since such a marker is considered to be deeply involved with the onset of an allergic disease, it may be an important target not only for diagnosis but also for the control of allergic symptoms.

### DISCLOSURE OF THE INVENTION

An objective of the present invention is to provide novel genes that can be used as allergy indicators, particularly for early stage allergic diseases. Another objective of the present invention is to provide methods of testing for early stage allergic diseases, and methods of screening candidate compounds for therapeutic agents for allergic diseases, both methods using the indicators.

The genes associated with early stage allergic diseases are located upstream of other genes associated with allergic symptoms, and may play the role of inducing the expression of these other genes. The present inventors hypothesized that if such genes could be identified, investigating their expression state would allow diagnosis of early stage allergic diseases.

Furthermore, the present inventors thought that such genes could also be used as important targets in treatment of allergic diseases. Effective pharmaceutical agents for early stage allergic diseases may be used as effective therapeutic agents against fundamental causes of pathology, not only at early stages of allergy but also after advancing to severe conditions. Pharmacological effects leading to complete cure of allergies instead of a mere symptomatic treatment can be expected from such therapeutic agents.

First, the present inventors isolated genes whose expression differed between the peripheral blood eosinophils obtained from healthy subjects and patients with atopic dermatitis. The differential display (DD) system (WO 00/65046) developed by the present inventors was applied to the method for obtaining a gene based on differences in expression levels. This a DD system is based on the previously established procedure of “Fluorescent DD method” (T. Ito et al., 1994, FEBS Lett. 351: 231-236), wherein leukocyte RNA samples prepared from the blood of a plurality of humans are analyzed. Eosinophils were selected as the target cells for the gene expression comparison. Eosinophils are important indicators for allergic symptoms. Therefore, genes whose expression levels differ in eosinophil cells are considered to be closely related to allergic symptoms.

Next, the present inventors compared the expression levels of genes obtained by the DD system in patients at different stages of advancement of allergic diseases and in healthy subjects. The inventors postulated that genes relating to early stage allergic disease could be found by selecting genes whose expression levels in eosinophils differ between patients with early stage allergic diseases and healthy subjects by comparing expression levels in patients at different stages of advancement of allergic diseases with those in healthy subjects.

As a result of analyzing the expression levels of genes in the peripheral blood eosinophils based on such strategy, the present inventors confirmed that the gene comprising the nucleotide sequence of SEQ ID NO: 1 showed significantly increased expression in the eosinophils of patients with early stage allergic diseases.

When a database search was performed on this nucleotide sequence, the nucleotide sequence of the 1835-17 fragment of SEQ ID NO: 1 was nearly identical to the nucleotide sequence (GenBank AF248540) predicted to encode intersectin 2. The relationship between the intersectin 2 gene and an allergic disease has not been suggested. Therefore, the finding of the present invention is novel. Furthermore, the present inventors found that testing for allergic diseases, and screening of candidate compounds for therapeutic agents for allergic diseases can be performed by using as an indicator the expression level of this gene, and completed this invention.

Specifically, the present invention relates to the use of a gene showing high expression level in early stage allergic disease as an indicator gene for allergic diseases. More specifically, this invention relates to a method for testing an allergic disease using the expression of the gene as an indicator, and a method for detecting the influence of a candidate compound on the expression of the gene, and additionally, a method of screening for a candidate compound for therapeutic agents for an allergic disease, which is based on this detection method.

[1] A method of testing for an allergic disease, said method comprising the steps of:

a) measuring the expression level of an indicator gene in a biological sample of a test subject; and

b) comparing the expression level of the indicator gene in the biological sample of a test subject to that of a healthy subject, wherein the indicator gene is intersectin 2 gene or a gene functionally equivalent thereto.

[2] The testing method of [1], wherein the allergic disease is atopic dermatitis.

[3] The testing method of [1], wherein the expression level of the gene is measured by cDNA PCR.

[4] The testing method of [1], wherein the expression level of the gene is measured by detecting a protein encoded by the gene.

[5] The method of [1], wherein the biological sample contains peripheral blood eosinophil cells.

[6] A reagent for testing for the presence of an allergic disease, which comprises an oligonucleotide containing at least 15 nucleotides of a nucleotide sequence complementary to a polynucleotide containing the nucleotide sequence of intersectin 2 gene or a gene functionally equivalent thereto or to a complementary strand of the polynucleotide.

[7] A reagent for testing for an allergic disease, which comprises an antibody that recognizes a peptide containing an amino acid sequence encoded by intersectin 2 gene or by a gene that is functionally equivalent thereto.

[8] A method of screening for a therapeutic agent for an allergic disease, said method comprising the steps of:

(1) contacting a candidate compound with cells expressing an indicator gene;

(2) measuring the expression level of the indicator gene; and

(3) selecting a compound that decreases the expression level of the indicator gene compared to a control,

wherein the indicator gene is intersectin 2 gene or a gene functionally equivalent thereto.

[9] The method of [8] wherein the cells are eosinophil cells.

[10] The method of screening for a therapeutic agent for an allergic disease, said method comprising the steps of:

(1) administering a candidate compound to a test animal;

(2) measuring the expression intensity of an indicator gene in a physiological sample of the test animal; and

(3) selecting a compound that decreases the expression level of the indicator gene compared to a control,

wherein the indicator gene is intersectin 2 gene or a gene functionally equivalent thereto.

[11] A method of screening for a therapeutic agent for an allergic disease, said method comprising the steps of:

(1) contacting a candidate compound with a cell transfected with a vector comprising a transcription regulatory region of an indicator gene and a reporter gene that is expressed under the control of the transcription regulatory region;

(2) measuring the activity of the reporter gene; and

(3) selecting a compound that decreases the expression level of the reporter gene compared to a control,

wherein the indicator gene is intersectin 2 gene or a gene functionally equivalent thereto.

[12] A method of screening for a therapeutic agent for an allergic disease, said method comprising the steps of:

(1) contacting a candidate compound with a protein encoded by an indicator gene;

(2) measuring the activity of the protein; and

(3) selecting a compound that decreases the activity of the protein, compared to a control,

wherein the indicator gene is intersectin 2 gene or a gene functionally equivalent thereto.

[13] A therapeutic agent for an allergic disease, which comprises as an active ingredient a compound obtainable by the screening method of any one of [8], [10], [11], and [12].

[14] A therapeutic agent for an allergic disease, which comprises an antisense DNA of an indicator gene or a portion thereof as the main ingredient, wherein the indicator gene is intersectin 2 gene or a gene functionally equivalent thereto.

[15] A therapeutic agent for an allergic disease, which comprises as the main ingredient an antibody that binds to a protein encoded by an indicator gene, wherein the indicator gene is intersectin 2 gene or a gene functionally equivalent thereto.

[16] Use of a transgenic non-human vertebrate as an animal model for an allergic disease, which has increased expression intensity of an indicator gene in eosinophil cells, wherein the indicator gene is intersectin 2 gene or a gene functionally equivalent thereto.

[17] A kit for screening for a therapeutic agent for an allergic disease, which comprises an oligonucleotide containing at least 15 nucleotides of a nucleotide sequence complementary to a polynucleotide containing the nucleotide sequence of an indicator gene or to a complementary strand of the polynucleotide, and a cell that expresses the indicator gene, wherein the indicator gene is intersectin 2 gene or a gene functionally equivalent thereto.

[18] A kit for screening for a therapeutic agent for an allergic disease, which comprises an antibody that recognizes a peptide comprising the amino acid sequence encoded by an indicator gene, and a cell that expresses the indicator gene, wherein the indicator gene is intersectin 2 gene or a gene functionally equivalent thereto.

The structure of the intersectin 2 gene used as an indicator gene of this invention, has already been revealed. More specifically, the nucleotide sequence of the 1835-17 fragment (SEQ ID NO: 1), which was isolated by the present inventors based on the DD method, mostly matched with the nucleotide sequence (KIAA1256; GenBank AB033082) that is predicted to encode intersectin 2. Therefore, the intersectin 2 gene can be used as an indicator gene in the context of this invention. Furthermore, Seifert, M. revealed the full length amino acid sequence (GenBank AF248540) encoded by the intersectin 2 gene. The nucleotide sequence (GenBank AF248540) of the intersectin 2 gene of this invention is shown in SEQ ID NO: 15, and the amino acid sequence encoded by this nucleotide sequence is shown in SEQ ID NO: 16.

The intersectin 2 gene (KIAA1256 gene) was isolated as a novel gene from a library derived from the human brain, and was registered in the DNA database (GenBank AB033082) by Ohara, O. et al. Since KIAA1256 gene and its amino acid have homology to the nucleotide sequence and amino acid sequence (GenBank Accession No. AF132480) of the previously registered mouse intersectin 2 (EMBO J. 18 (5), 1159-1171 (1999)), it is predicted to be human intersectin 2. GenBank Accession No. AF132480 has been registered under the name of Mouse ESE2. Human intersectin 2 and mouse intersectin 2 are highly homologous (nucleotide sequence level: 89%; amino acid sequence level: 94%). As confirmed in the Examples, expression of mouse intersectin 2 was found to increase in an allergic dermatitis model. Therefore, a polynucleotide comprising a nucleotide sequence having 90% or higher identity to the nucleotide sequence of SEQ ID NO: 15 can be used as an indicator gene of this invention. Alternatively, a polynucleotide encoding an amino acid sequence having 95% or higher identity to the amino acid sequence of SEQ ID NO: 16 can be used as an indicator gene of this invention.

The present invention relates to the use of intersectin 2 gene or a gene functionally equivalent thereto as an indicator for an allergic disease. In the present invention, intersectin 2 gene and genes functionally equivalent thereto are collectively referred to as indicator genes. Furthermore, the protein encoded by an indicator gene is referred to as an indicator protein. The structure of the intersectin 2 gene is known as GenBank Accession No. AF248540.

In the present invention, when a certain protein shows increased expression in eosinophils of a patient with an early stage allergic disease or an animal with an early stage allergic disease, it is said to be functionally equivalent to the protein having the amino acid sequence of SEQ ID NO: 16. Increase in expression of a certain protein in eosinophils can be confirmed by comparing the expression levels of the gene encoding the protein in collected eosinophils.

In the present invention, the functionally equivalent gene includes a polynucleotide that encodes a functionally equivalent protein and hybridizes under stringent conditions to a polynucleotide comprising the nucleotide sequence of SEQ ID NO: 15. Such a polynucleotide can be obtained by known methods, such as hybridization and PCR, based on the nucleotide sequence of SEQ ID NO: 15. For example, cDNA comprising a nucleotide sequence having high identity to the nucleotide sequence of SEQ ID NO: 15 can be obtained by screening a cDNA library of leukocytes under stringent conditions using as a probe an oligonucleotide comprising any nucleotide sequence selected from the nucleotide sequence of SEQ ID NO: 15. When a certain polynucleotide hybridizes under stringent conditions with a polynucleotide comprising the nucleotide sequence of SEQ ID NO: 15, many of the proteins encoded by this polynucleotide are considered to have activity similar to the indicator protein.

Stringent conditions mean hybridization in 4×SSC at 65° C. followed by washing with 0.1×SSC at 65° C. for 1 hour. Temperature conditions for hybridization and washing that greatly influence stringency can be adjusted according to the melting temperature (Tm) Tm varies with the ratio of constitutive nucleotides in the hybridizing base pairs, and the composition of hybridization solution (concentrations of salts, formamide, and sodium dodecyl sulfate). Therefore, considering these conditions, those skilled in the art can select an appropriate condition to produce an equal stringency from their experience.

A protein encoded by cDNA comprising the nucleotide sequence that has a high identity to the cDNA of this invention would be a functionally equivalent protein in this invention. Herein, a nucleotide sequence with a high identity refers to a nucleotide sequence that shows 70% or more homology in general, usually 80% or more, preferably 90% or more, more preferably 95% or more, furthermore preferably 98% or more, and specifically preferably 99% or more identity with a nucleotide sequence of this invention. The degree of identity of one nucleotide sequence to another can be determined by following the well-known algorism such as BLASTN.

Alternatively, a gene encoding a protein having, for example, 90% or more, preferably 95% or more, and furthermore preferably 99% or more homology to the amino acid sequence (SEQ ID NO: 16) of intersectin 2 protein can be referred to as a gene functionally equivalent to the intersectin 2 gene, respectively.

Alternatively, a cDNA with a high identity to a cDNA of this invention can be obtained by PCR performed using oligonucleotides comprising a nucleotide sequence selected from the nucleotide of SEQ ID NO: 15 as the primers and a leukocyte cDNA library as a template. If human cells are used as a source of cDNA, it is possible to obtain human cDNA. When cells from vertebrates other than humans are used, it is possible to obtain the counterpart of human cDNA in different animal species. Examples of such non-human animals include various experimental animals such as mice, rats, dogs, pigs, and goats. For example, mouse intersectin 2 is well known. An indicator gene of this invention in an experimental animal is useful in preparing allergic disease animal models from various animal species and as the marker in developing therapeutic agents for allergic diseases.

A gene that can be amplified using, as primer, an oligonucleotide comprising a nucleotide sequence selected from the nucleotide sequence of SEQ ID NO: 15 and that encodes a protein whose expression significantly increases in eosinophils of patients with early stage allergic diseases is also a functionally equivalent gene.

Herein, the term “allergic disease” is a general term for diseases in which allergic reaction is involved. More specifically, it is defined as a disease in which an allergen must be identified, a strong correlation between the exposure to the allergen and the onset of the pathological change must be demonstrated, and the pathological change must be proven to have an immunological mechanism. Herein, an immunological mechanism means that immune responses by the leukocytes are induced by the stimulation of the allergen. Examples of known allergens include mite antigen, and pollen antigen.

Representative allergic diseases include atopic dermatitis, bronchial asthma, allergic rhinitis, pollen allergy, and insect allergy. Allergic diathesis is a genetic factor that is inherited from allergic parents to their children. Familial allergic diseases are also called atopic diseases, and the causative factor that is inherited is the atopic diathesis.

The indicator gene of this invention showed increased expression level in the eosinophils of patients with light atopic dermatitis compared to those of healthy subjects. Therefore, allergic diseases can be tested using the expression levels of the indicator gene of this invention as indicators. In the test method of this invention, not only the indicator gene of this invention, but other indicators for allergic disease can be used in combination. The testing based on a plurality of indicators allows a more accurate determination. Since a group of patients with allergic disease such as atopic dermatitis is heterogeneous, more reliable diagnosis can be performed by using a multiple indicator genes.

For example, the testing method for diagnosing an allergic disease of this invention includes the following methods. Specifically, an increase in the expression level of the indicator gene of this invention in a patient showing early symptoms suspect of an allergic disease, proves that the early symptoms in that patient is caused by an allergic disease.

Herein, the expression level of the indicator gene of this invention includes the transcription of the gene to mRNA as well as the translation into a protein. Therefore, a method for testing for allergic disease according to the present invention may be performed by comparing either the expression intensity of mRNAs corresponding to the genes, or the expression levels of a proteins encoded by the genes.

Measurement of the expression level of the indicator gene in a test for allergic diseases of the present invention may be conducted according to known gene analytical methods. More specifically, for example, a hybridization technique with nucleic acids that hybridize to the genes as a probe, a gene amplification technique with DNA hybridizing to the indicator gene as a primer, or such can be utilized.

A polynucleotide that has at least 15 nucleotides and that is complementary to a polynucleotide comprising the nucleotide sequence of SEQ ID NO: 15 or the complementary strand thereof can be used as a primer or probe for the test according to the present invention. Herein, the term “complementary strand” means one strand of a double stranded DNA composed of A:T (U for RNA) and G:C base pairs to the other strand. In addition, “complementary” encompasses both those nucleotides completely complementary to a region of at least 15 continuous nucleotides, as well as those having a homology of at least 70%, preferably at least 80%, more preferably 90%, and even more preferably 95% or higher. The degree of homology between nucleotide sequences can be determined by the algorithm, such as BLAST.

Such polynucleotides can be useful as the probe to detect and isolate the polynucleotide encoding the indicator protein, or as the primer to amplify the polynucleotide according to the present invention. When used as a primer, those polynucleotides comprise usually 15 bp to 100 bp, preferably 15 bp to 35 bp of nucleotides. When used as a probe, DNAs comprising the whole sequence of a polynucleotide according to the present invention, or a partial sequence thereof that contains at least 15-bp nucleotides may be used. When used as a primer, the 3′ region thereof must be complementary to the indicator gene, while the 5′ region can be linked to a restriction enzyme-recognition sequence or tag.

The “polynucleotides” of the present invention may be either DNA or RNA. These polynucleotides may be either synthetic or naturally-occurring. Also, DNAs used as probes for hybridization are usually labeled. Examples of labeling methods include those as described below. Herein, the term “oligonucleotide” means a polynucleotide with relatively low degree of polymerization. Oligonucleotides are included in polynucleotides.

nick translation labeling using DNA polymerase I;

end labeling using polynucleotide kinase;

fill-in end labeling using Klenow fragment (Berger, S L, Kimmel, A R. (1987) Guide to Molecular Cloning Techniques, Method in Enzymology, Academic Press; Hames, B D, Higgins, S J (1985) Genes Probes: A Practical Approach. IRL Press; Sambrook, J, Fritsch, E F, Maniatis, T. (1989) Molecular Cloning: a Laboratory Manual, 2nd Edn. Cold Spring Harbor Laboratory Press);

transcription labeling using RNA polymerase (Melton, D A, Krieg, P A, Rebagkiati, M R, Maniatis, T, Zinn, K, Green, M R. (1984) Nucleic Acid Res., 12, 7035-7056); and

non-isotopic labeling of DNA by incorporating modified nucleotides (Kricka, L J. (1992) Nonisotopic DNA Probing Techniques. Academic Press).

For testing for the presence of an allergic disease using hybridization techniques, for example, Northern hybridization, dot blot hybridization, or DNA microarray technique may be used. Furthermore, gene amplification techniques, such as RT-PCR method, may be used. By using the PCR amplification monitoring method during the gene amplification step in RT-PCR, one can achieve more quantitative analysis for the expression of the indicator gene.

In the PCR gene amplification monitoring method, the detection target (DNA or reverse transcript of RNA) is hybridized to probes that are dual-labeled at both ends with different fluorescent dyes whose fluorescences cancel each other out. When the PCR proceeds and Taq polymerase degrades the probe with its 5′-3′ exonuclease activity, the two fluorescent dyes become distant from each other and the fluorescence becomes to be detected. The fluorescence is detected in real time. By simultaneously measuring a standard sample in which the copy number of the target is known, it is possible to determine the copy number of the target in the subject sample with the cycle number where PCR amplification is linear (Holland, P. M. et al., 1991, Proc. Natl. Acad. Sci. USA 88: 7276-7280; Livak, K. J. et al., 1995, PCR Methods and Applications 4 (6): 357-362; Heid, C. A. et al., 1996, Genome Research 6: 986-994; Gibson, E. M. U. et al., 1996, Genome Research 6: 995-1001). For the PCR amplification monitoring method, for example, ABI PRISM7700 (PE Biosystems) may be used.

The method of testing for allergic diseases of the present invention can also be carried out by detecting a protein (indicator protein) encoded by the indicator gene of this invention. Such test methods include, for example, those utilizing antibodies binding to a protein encoded by such a gene, including the Western blotting method, the immunoprecipitation method, and the ELISA method.

Antibodies that bind to the indicator proteins used in the detection may be produced by techniques known to those skilled in the art. Antibodies used in the present invention may be polyclonal or monoclonal antibodies (Milstein, C. et al., 1983, Nature 305 (5934): 537-40). For example, polyclonal antibodies against the indicator proteins may be produced by collecting blood from mammals sensitized with an antigen, and separating the serum from this blood using known methods. As polyclonal antibodies, the serum containing polyclonal antibodies may be used. According to needs, a fraction containing polyclonal antibodies can be further isolated from this serum. Alternatively, a monoclonal antibody can be obtained by isolating immune cells from mammals sensitized with an antigen; fusing these cells with myeloma cells, and such; cloning hybridomas thus obtained; and collecting the antibody from the culture as the monoclonal antibody.

To detect the indicator protein, these antibodies may be appropriately labeled. Alternatively, instead of labeling the antibodies, a substance that specifically binds to antibodies, for example, protein A or protein G, may be labeled to arrange an indirect detection of the proteins. More specifically, one example of an indirect detection method is ELISA.

A protein or partial peptides thereof that is used as an antigen may be obtained, for example, by inserting the gene or portion thereof into an expression vector, introducing it into an appropriate host cell to produce a transformant, culturing the transformant to express the recombinant protein, and purifying the expressed recombinant protein from the culture or the culture supernatant. Alternatively, oligonucleotide consisting of the amino acid sequence (SEQ ID NO: 16) encoded by the gene or partial amino acid sequence thereof is chemically synthesized to be used as the antigen.

Furthermore, in the present invention, testing for an allergic disease can be performed using as an index not only the expression level of the indicator gene but also the activity of the indicator protein in the biological sample. In the context of the present invention, the activity of the indicator protein refers to a biological activity intrinsic to the protein. The detection of activity of the indicator protein can be achieved by any known method.

For example, it has been revealed that mouse intersectin 2 (ESE2) is a protein involved in the uptake of a foreign substance into a cell via receptors on leukocytes or other cells. This action is generally called clathrin-mediated endocytosis. For example, when a cell incorporates a foreign substance by phagocytosis or pinocytosis, ESE2 associates with at least 14 other proteins or more to form a complex. Epsin, dynamin, and such are considered to bind to the EH and SH3 domains of ESE2 to mediate information for forming a vacuole by invaginating the cell membrane. Epsin, dynamin, and such are known to be proteins essential for phagocytosis. However, when ESE is overexpressed, phagocytotic ability is suppressed because complex formation does not proceed well.

In this invention, eosinophil cells of a test subject are used as the sample. Eosinophil cells can be prepared by conventional methods from the peripheral blood. Specifically, leukocytes can be isolated, for example, by fractionating heparinized blood by centrifugation. Next, granulocytes can be fractionated, for example, by Ficoll centrifugation of leukocytes, and furthermore eosinophil cells can be isolated, for example, by depletion of neutrophils using the CD16 antibody. A sample for immunological assays of the aforementioned protein can be obtained by disrupting the isolated eosinophils to produce a lysate. Alternatively, a sample for measuring mRNA corresponding to the aforementioned gene can be obtained by extracting mRNA from this lysate. The use of a commercially available kit is convenient for extracting mRNA and preparing a lysate of eosinophils.

Alternatively, the expression level of a gene that serves as an indicator in this invention may be measured not in isolated in eosinophils, but in the whole blood, and peripheral blood leukocyte population. In this case, by correcting the measured values, the change of gene expression levels in cells can be determined. For example, based on the measured value of the expression level of a gene (housekeeping gene), whose expression level is eosinophil specific and is not widely altered regardless of the cellular conditions, the measured value of the expression level of the gene serving as an indicator in this invention can be corrected.

Alternatively, in the case where the protein to be detected is a secretory protein, comparison of the expression level of a gene encoding the protein is accomplished by measuring the amount of the target protein contained in body fluid sample, such as blood and serum, in a subject.

In the method of testing for an allergic disease of this invention, the gene that shows increased expression in light allergic diseases is used as an indicator for early symptoms of an allergic disease.

A polynucleotide and an antibody necessary for various test methods of the present invention which are used for measuring the expression level of the indicator gene are useful as reagents for testing allergic diseases. As a reagent for measuring the expression level of the indicator gene, for example, an oligonucleotide that has at least 15 nucleotides complementary to the polynucleotide comprising the nucleotide sequence (SEQ ID NO: 15) of the indicator gene or to the complementary strand thereof may be used. Alternatively, an antibody that recognizes a peptide comprising amino acid sequence (SEQ ID NO: 16) of the indicator protein may be used as a reagent.

An oligonucleotide comprising a nucleotide sequence complementary to the polynucleotide having the nucleotide sequence of SEQ ID NO: 15 or to a complementary strand thereof, and that are at least 15-nucleotide-long, can be used as the oligonucleotide as described above. Herein, the term “complementary strand” is defined as one strand of a double stranded polynucleotide composed of A:T (U for RNA) and G:C base pairs to the other strand. In the context of the present invention, “complementary” strands need not be completely homologous within a region of at least 15 continuous nucleotides, provided they have at least 70%, preferably at least 80%, more preferably 90%, and even more preferably 95% or higher homology within that region. The degree of homology of one nucleotide sequence to another can be determined by known algorithms, such as BLAST.

These test reagents can be made into a kit for testing for an allergic disease in combination, for example, with a substrate compound used for detection of the label, a buffer for diluting the sample, or a positive or negative standard sample. Furthermore, an instruction sheet and such indicating the method of using the kit can be packaged in the kit for the testing of this invention.

According to this invention, the expression level of the aforementioned indicator gene in eosinophil cells was found to increase in eosinophils of patients with early stage atopic dermatitis. Therefore, animals in which the expression level of the gene or a gene that is functionally equivalent thereto in eosinophil cells is artificially enhanced can be utilized as animal models for early stage allergic diseases. The phrase “increase of the expression level of indicator genes in eosinophils” includes increase of their expression level in the entire population of leukocytes. That is, the expression level of the aforementioned genes may be increased not only in eosinophils alone, but also in the entire population of leukocytes. The functionally equivalent gene of this invention refers to a gene encoding a protein having activities similar to known activities of a protein encoded by the indicator gene. A representative example of a functionally equivalent gene is a counterpart of an indicator gene originally present in the animal species of the transgenic animal.

The genes showing increased expression in early stage allergic diseases can be said to be genes that regulate, at the upstream position, the pathology of allergic diseases. In other words, pathology of allergies is considered to appear when genes that act in early stage allergic diseases regulate expression or suppression of various genes positioned downstream therefrom. Thus, genes that show increased expression in early stage allergic diseases can be considered to be genes that play an important role in pathologic formation of allergies. Therefore, in allergy therapy, pharmaceutical agents that suppress the expression or inhibit the activity of these genes can be expected to have the function of not only simply improving allergic symptoms, but also eliminating the fundamental cause of pathologic formation of allergies.

As described above, a gene the expression level of which is increased in early stage allergic disease is very important. Therefore, it is highly significant to assess the role of the gene and the effects of drugs targeting this gene using transgenic animals, which can be obtained by elevating the expression level of this gene in vivo, as the early stage allergic disease model animal.

Early stage allergic disease model animals according to the present invention are useful in not only in screening drugs for treating or preventing early stage allergic diseases as described below but also in elucidating mechanisms of early stage allergic diseases, furthermore, testing the safety of compounds screened.

For example, if early stage allergic disease animal models according to the present invention either develop clinical manifestations of dermatitis or show changes in measured values related to any allergic disease, it is possible to construct a screening system to find a compound having activity to recover normal conditions.

In the present invention, an increase in the expression level means the state wherein a target gene is transduced as a foreign gene and forcibly expressed; the state wherein transcription of a gene inherent in the host and translation thereof into protein are increased; or the state wherein decomposition of the translation product, protein, is suppressed. Gene expression levels can be confirmed by, for example, the quantitative PCR as described in Examples. Furthermore, the activity of a translation product, protein, can be confirmed by comparing to that of an normal intersectin.

A typical transgenic animal is the one to which a gene of interest is transduced to be forcibly expressed. Examples of other types of transgenic animals are those in which a mutation is introduced into the coding region of the gene to increase its activity or to modify the amino acid sequence of the gene product protein so as to be hardly decomposed. Examples of mutations in the amino acid sequence include the substitution, deletion, insertion, or addition of amino acid(s). In addition, by mutagenizing the transcriptional regulatory region of the gene, the expression itself of the indicator gene can be controlled.

Methods for obtaining transgenic animals with a particular gene as a target are well known in the art. That is, a transgenic animal can be obtained by a method wherein the gene and ovum are mixed and treated with calcium phosphate; a method where the gene is introduced directly into the nucleus of oocyte in pronuclei with a micropipette under a phase contrast microscope (microinjection method, U.S. Pat. No. 4,873,191); or a method wherein embryonic stem cells (ES cells) are used. Furthermore, there have been developed methods for infecting ovum with a gene-inserted retrovirus vector, a sperm vector method for transducing a gene into ovum via sperm, and such. The sperm vector method is a gene recombination technique for introducing a foreign gene by fertilizing ovum with sperm after a foreign gene has been incorporated into sperm by the adhesion or electroporation method, and so on (M. Lavitranoet, et al. Cell, 57, 717, 1989).

Transgenic animals used as animal models for early stage allergic diseases of the present invention can be produced using all the vertebrates except for humans. More specifically, transgenic animals having various transgenes and having modified gene expression levels thereof can be produced using vertebrates such as mice, rats, rabbits, miniature pigs, goats, sheep, or cattle.

Furthermore, this invention relates to a method of screening for a therapeutic agent for an allergic disease. In this invention, the indicator gene shows significant increase in expression in the eosinophils of patients with light atopic dermatitis. Therefore, based on the method of detecting an influence on the expression level of the gene, a therapeutic agent for an allergic disease can be obtained by selecting a compound that can decrease the expression level of the gene. In the present invention, a compound that decreases the expression level of the gene is a compound having the effect of inhibiting any one of the steps of transcription of the gene, translation, and expression of protein activity.

A method of screening for a therapeutic agent for an allergic disease of this invention can be carried out either in vivo or in vitro. This screening method can be carried out, for example, according to the steps as described below:

(1) administering a candidate compound to a test animal;

(2) measuring the expression level of the indicator gene in an eosinophil cell from the test animal; and

(3) selecting a compound that reduces the expression level of the indicator gene, as compared to a control.

More specifically, a screening method according to the present invention can be carried out by comparing the expression level of the indicator gene in the biological sample collected from a test animal to that in a control. Examples of suitable biological samples include whole blood, peripheral blood mononuclear cells (PBMC), and eosinophil cells. Methods for collecting and preparing these biological samples are known.

For example, as a model closely resembling human atopic dermatitis, a spontaneous dermatitis NC/Nga mouse model has been reported. When mite antigen is administered to the auricles of this mouse (5 μg/ear) eight times at intervals of 2 to 3 days, symptoms strikingly similar to human atopic dermatitis can be induced after two weeks. The-screening of this invention can be carried out by administering a candidate compound to this mouse and monitoring changes in the expression level of the indicator gene.

In this manner, the influence of the candidate compound for a pharmaceutical agent on the expression level of the indicator gene can be detected by administering the candidate compound to a model animal expressing the indicator gene, and monitoring the effect of the compound on expression of the indicator gene in eosinophils of the model animal. Furthermore, based on the results of this detection, the candidate compound for a pharmaceutical agent can be screened by selecting a candidate compound for a pharmaceutical agent that reduces the expression level of the indicator gene, compared to the control.

Such screening allows for the selection of drugs that are involved in various ways in the expression of the indicator gene. Specifically, for example, a candidate compound for a pharmaceutical agent having the following action can be discovered:

Suppression of a signal transduction pathway that causes expression of indicator gene of this invention;

Suppression of transcription activity of indicator gene of this invention; and

Facilitation of degradation of the transcription product of indicator gene of this invention.

Examples of in vitro screening include a method in which cells expressing an indicator gene are contacted with a candidate compound to select a compound that reduces the expression level of the indicator gene. This screening may be carried out, for example, according to the steps of:

(1) contacting a candidate compound with cells expressing an indicator gene;

(2) measuring the expression level of the indicator gene; and

(3) selecting a compound that reduces the expression level of the candidate gene, as compared to a control.

In this invention, cells to be used in the step (1) can be obtained by inserting an indicator gene into an appropriate expression vector and then transfecting suitable host cells with the vector. Any vectors and host cells may be used, so long as they are capable of expressing the indicator gene. Examples of host cells in the host-vector system are Escherichia coli cells, yeast cells, insect cells, animal cells, and available vectors usable for each can be selected.

Vectors may be transfected into the host by biological methods, physical methods, chemical methods, and so on. Examples of biological methods include methods using virus vectors; methods using specific receptors; the cell-fusion method (HVJ (Sendai virus) method; the polyethylene glycol (PEG) method; the electric cell fusion method, and microcell fusion method (chromosome transfer)). Examples of physical methods include the microinjection method, the electroporation method, and the method using gene particle gun. The chemical methods are exemplified by the calcium phosphate precipitation method, the liposome method, the DEAE-dextran method, the protoplast method, the erythrocyte ghost method, the erythrocyte membrane ghost method, and the microcapsule method.

In the screening method as described above, leukocyte cell lines can be used as cells for expressing an indicator gene. Examples of leukocyte cell lines are cell lines derived from leukocytes, such as Eol, YY-1, HL-60, TF-1, and AML14.3D10. Among the leukocyte cell lines, cell lines derived from eosinophils are preferred for the screening method of this invention. The following are cell lines derived from eosinophils:

Eol;

YY-1; and

AML14.3D10.

Eol (Eol-1: Saito H et al., Establishment and characterization of a new human eosinophilic leukemia cell line. Blood 66, 1233-1240, 1985) can be obtained from Hayashibara Research Institute. Similarly, YY-1 (Ogata N et al., The activation of the JAK2/STAT5 pathway is commonly involved in signaling through the human IL-5 receptor. Int. Arch. Allergy Immunol., Suppl 1, 24-27, 1997) is available from The Institute of Cytosignal Research. Furthermore, AML14.3D10 (Baumann M A et al., The AML14 and AML14.3D10 cell lines: a long-overdue model for the study of eosinophils and more. Stem Cells, 16, 16-24, 1998) is commercially available from Paul CC at Research Service, VA Medical Center, Dayton, Ohio, USA.

In addition, by culturing in the presence of butyric acid for about 1 week, HL-60 clone 15 (ATCC CRL-1964), which is an undifferentiated leukocyte cell line, can differentiate into eosinophils to give an eosinophil cell line. Eosinophils can be detected due to their morphological characteristic of being polymorphonuclear and having eosinophilic granules. Morphological observations are performed by Giemsa staining and Difquick staining. Generally, human leukocyte cell lines including eosinophils can be established by cloning immortalized cells from a leukemia patient sample. Therefore, those skilled in the art can obtain eosinophil cell lines by a conventional method when necessary.

The screening method of the present invention first involves contacting a candidate compound with the aforementioned leukocyte cell line. Then, the expression level of the indicator gene in the leukocyte cell line is measured and a compound that decreases the expression level of the gene is selected.

In the screening method of the present invention, expression level of the indicator gene can be compared by detecting the expression level of not only a protein encoded by the gene but also the corresponding mRNA. For the comparison of the expression level using mRNA, the step of preparing mRNA sample as described above is conducted in place of the step of preparing a protein sample. Detection of mRNA and protein can be carried out according to the known methods as described above.

Furthermore, it is possible to obtain the transcriptional regulatory region of the indicator gene and to construct a reporter assay system. In the context of the present invention, a reporter assay system refers to an assay system for screening for a transcriptional regulatory factor that acts on the transcriptional regulatory region by using the expression level of a reporter gene that is located downstream of the transcriptional regulatory region and expressed under the control of the regulatory region as an indicator.

More specifically, this invention relates to a method of screening for therapeutic agents for an allergic disease, the method comprising the steps of:

(1) contacting a candidate compound with a cell transfected with a vector containing the transcription regulatory region of an indicator gene and a reporter gene that is expressed under the control of this transcription regulatory region;

(2) measuring-the activity of the reporter gene; and

(3) selecting a compound that decreases the expression level of the reporter gene compared to a control,

wherein the indicator gene is an intersectin 2 gene or a gene functionally equivalent to intersectin 2.

A transcriptional regulatory region is exemplified by promoter, enhancer, as well as CAAT box, and TATA box, which are usually found in the promoter region. Examples of reporter genes include the chloramphenicol acetyltransferase (CAT) gene, the luciferase gene, and growth hormone genes.

A transcriptional regulatory region of an indicator gene can be obtained as follows. Specifically, first, based on the nucleotide sequence of an indicator gene, a human genomic DNA library, such as BAC library and YAC library, is screened by a method using PCR or hybridization to obtain a genomic DNA clone containing the sequence of the cDNA. Based on the sequence of the resulting genomic DNA, the transcriptional regulatory region of an indicator gene can be predicted and obtained. The obtained transcriptional regulatory region is cloned so as to be localized upstream of a reporter gene to prepare a reporter construct. The resulting reporter construct is introduced into a cultured cell strain to prepare a transformant for screening. By contacting a candidate compound with this transformant to detect the expression of a reporter gene, it is possible to assess the effect of the candidate compound on the transcriptional regulatory region.

Based on the method of detecting the effect on the expression level of the indicator gene, it is possible to carry out screening for a compound that alters the expression level of the indicator gene. This invention relates to a method of screening for a compound that alters the expression level of the indicator gene, comprising following steps.

That is, the present invention relates to a method of screening for a compound that decreases the expression level of an indicator gene, the method comprising the steps of detecting the effect of a candidate compound on the expression level of the indicator gene in vivo and/or in vitro, and selecting a compound that raises the expression level as compared to a control.

Alternatively, this invention relates to a method of screening for a compound that acts on the transcriptional regulatory region by the reporter assay utilizing the transcriptional regulatory region of the indicator gene. Based on the results of reporter assay according to this invention, by selecting a compound that decreases the expression level of the reporter gene as compared to a control, it is possible to obtain a compound that suppresses the expression of the indicator gene.

As an in vitro screening method of this invention, a screening method based on activities of indicator proteins can be used. That is, this invention relates to a method of screening for a therapeutic agent for an allergic disease, in which the indicator genes are intersectin 2 gene or genes that are functionally equivalent thereto, the method comprising the following steps:

(1) contacting the candidate substance with one or more proteins encoded by the indicator genes;

(2) measuring the activity of the aforementioned proteins; and

(3) selecting a compound that lowers the activity of the aforementioned proteins, as compared to a control.

Intersectin 2, which is the indicator protein of this invention, has ESE activities. Using these activities as indicators, compounds that inhibit such activities can be screened.

The ESE activities can be evaluated by measuring clathrin-mediated endocytosis. The method for measuring clathrin-mediated endocytosis (Mol. Biol. Cell, 8, 2003-2015, 1997) using the biological response of a cell as an indicator, and such are well known. As a conventional method for evaluating ESE activities, the protocol for transferrin uptake assay is described below.

First, a cell transformed with a vector that expresses a test protein is prepared. An example of suitable cells is COS7 cells. The transformed COS7 cells are incubated in serum-free Dulbecco's modified Eagle Medium (DMEM) for about 36 hours. Next, the cells are cultured for about 1 hour in DMEM containing biotin-labeled transferrin (25 μg/mL). The level of transferrin uptake by the cell after cultivation is evaluated by visualizing biotin using avidin-labeled isothiocyanate and such. Furthermore, by expressing the test protein as a fused protein with a MYC tag and such, localization of the test protein can be evaluated. Localization of the test protein and biotin-labeled transferrin in the same part indicates that the test protein enhances ferritin uptake. Namely, the test protein is evaluated to have ESE activity.

In order to perform the screening of this invention using the above-mentioned evaluation method, the cell is contacted with the candidate substance prior to addition of biotin-labeled transferrin, or the biotin-labeled transferrin is contacted with the cell in the presence of the candidate substance. If the transferrin uptake level changes due to contact with the candidate substance, the candidate substance is judged to have the activity to change the activity of the protein. The screening method of this invention can be carried out by selecting a compound that decreases transferrin uptake as compared to a control, in which the cell has not been contacted with the candidate substance.

A compound obtainable by this method suppresses the function of intersectin 2. Hence, it is able to regulate allergic immune response through inhibition of the indicator protein whose expression is induced in eosinophil cells.

The polynucleotide, antibody, cell line, or model animal, which are necessary for the various methods of screening of this invention, can be combined in advance to produce a kit. More specifically, such a kit may comprise, for example, a cell that expresses the indicator gene, and a reagent for measuring the expression level of the gene. As a reagent for measuring the expression level of the indicator gene, for example, an oligonucleotide that has at least 15 nucleotides complementary to the polynucleotide comprising the nucleotide sequence of at least one indicator gene or to the complementary strand thereof may be used. Alternatively, an antibody that recognizes a peptide comprising amino acid sequence of at least one indicator protein may be used as a reagent.

In these kits may be packaged a substrate compound used for the detection of the indicator, medium and a vessel for cell culturing, positive and negative standard samples, and furthermore, a manual describing how to use the kit. A kit of this invention, for detecting the effect of a candidate compound on the expression level of the indicator gene of this invention, can be used for screening for a compound that modifies the expression level of the indicator gene of this invention.

Test candidate compounds used in these methods include, in addition to compound preparations synthesized by known chemical methods, steroid derivatives and compound preparations synthesized by combinatorial chemistry, and mixtures of multiple compounds such as extracts from animal or plant tissues, or microbial cultures and their purified preparations.

Compounds selected by the screening method of this invention are useful as therapeutic agents for an allergic disease. The expression level of the indicator gene of this invention is increased in eosinophils of patients with early stage allergic diseases. Accordingly, a compound capable of decreasing the expressions of the genes is expected to suppress symptoms of atopic dermatitis. A therapeutic agent for one or more allergic diseases of the present invention can be formulated by including a compound selected by the screening methods as the effective ingredient, and mixing with a physiologically acceptable carrier, excipient, diluent, and such. To ameliorate allergic symptoms, the therapeutic agent for allergic diseases of this invention can be administered orally or parenterally.

Oral drugs can take any dosage form including granules, powder, tablets, capsules, solution, emulsion, suspension, and so on. Injections include subcutaneous injection, intramuscular injection, and intraperitoneal injection.

Furthermore, for administering a compound that is composed of protein, a therapeutic effect can be achieved by introducing a gene encoding the protein into the living body using gene therapeutic techniques. The techniques for treating disease by introducing a gene encoding a therapeutically effective protein into the living body and expressing it therein are well known in the art.

Alternatively, an antisense DNA can be incorporated downstream of an appropriate promoter sequence to be administered as an antisense RNA expression vector. When this expression vector is introduced into eosinophil cells of an allergic disease patient, a therapeutic effect on allergic disease is achieved by the reduction of the expression level of the gene through the expression of the corresponding antisense gene. For introducing the expression vector into eosinophil cells, methods performed either in vivo or ex vivo are known.

Furthermore, compounds that inhibit the activity of proteins (i.e. indicator proteins) that are expression products of the indicator genes of this invention, are also expected to show therapeutic effects on allergies. For example, antibodies that recognize the indicator proteins of this invention and suppress their activity are useful as pharmaceutical agents for treatment of allergic diseases. Methods for preparing antibodies that suppress protein activity are well known. For administration to humans, antibodies may be prepared as chimeric antibodies, humanized antibodies, or human-type antibodies to serve as highly safe pharmaceutical agents.

Although the dosage may vary depending on the age, sex, body weight, and symptoms of a patient; treatment effects; method for administration; treatment duration; type of active ingredient contained in the drug composition; and such, a range of 0.1 to 500 mg, preferably 0.5 to 20 mg per dose for an adult can be administered. However, the dose changes according to various conditions, and thus in some case a smaller amount than that mentioned above is sufficient whereas an amount above the above-mentioned range is required in other cases.

### BRIEF DESCRIPTION OF THE DRAWINGS

FIG. 1 is a graph showing the distribution of the numbers of peripheral blood eosinophils (cells/μL) in healthy subjects and in patients with various atopic dermatitis symptoms.

FIG. 2 is a graph showing the distribution of total IgE concentrations (UA/mL) in healthy subjects and in patients with various atopic dermatitis symptoms.

FIG. 3 is a graph showing the distribution of the 1835-17 (intersectin 2) gene expression levels (copy/ng RNA) in healthy subjects and in patients with various atopic dermatitis symptoms.

FIG. 4 is a graph showing the expression levels of the 1835-17 (intersectin 2) gene (copy/ng RNA, GAPDH corrected value) in the peripheral blood eosinophils of a healthy subject in the presence of various cytokines indicated along the horizontal axis.

FIG. 5 is a graph showing the comparison of expression levels of the 1835-17 (intersectin 2) gene in a model of inflammatory allergic reaction induced by 2,4-dinitrofluorobenzene (DNFB) administration. The vertical axis shows the relative expression level (Relative activity) calculated taking the quantitative value of mRNA corrected with the 18s gene of the control (CONT.) as 1. The horizontal axis shows the types of models used in the experiment.

### BEST MODE FOR CARRYING OUT THE INVENTION

The present invention is explained in detail below with reference to examples, but should not to be construed as being limited thereto.

### EXAMPLE 1

### Differential Display Analysis

Screening was performed to discover novel genes relating to therapy or useful for diagnosis, which show varying expression when comparing hemocytes isolated from the peripheral blood of a healthy patient to those of an atopic dermatitis patient.

(1) Subjects:

Symptoms, pathology, presence of asthma, mite-specific IgE values, numbers of eosinophils, and total IgE values of healthy subjects (lanes 1 to 6) and those with atopic dermatitis (lanes 8 to 29) are shown in Table 1. Allergen non-specific (Total IgE), mite-specific, and cedar-specific IgEs were measured by the EIA method. More specifically, the test sera were allowed to react to an anti-human IgE antibody-bound cap to bind thereto allergen non-specific IgE antibody or mite- or cedar-specific IgE antibodies in the sera. Next, β-D-galactosidase-labeled anti-human IgE antibody and a substrate solution (4-methylumbelliferyl-β-D-galactopyranoside) were added and allowed to react to produce a fluorescent substance. The reaction was quenched by adding a quenching solution, and the antibody concentration was determined from the fluorescence intensity of a simultaneously measured standard IgE. LDH was measured by the UV method (Wroblewski-La Due method) and the rate of decrease of NADH caused by the reaction of pyruvic acid with NADH is calculated from decrease in absorbance. L-type Wako LDH (Wako Pure Chemicals) and 7170-type automatic analyzer (Hitachi) were used for measuring the LDH values. The number of eosinophils was measured by microscopic examination and automatic hemocyte analyzer SE-9000 (RF/DC impedance system, Sysmex) using 2 ml of EDTA-added blood as the sample.

TABLE 1

| Lane | 1 | 2 | 3 | 4 | 5 | 6 | 8 | 9 | 10 | 11 | 12 | 14 | 18 | 19 | 24 | 26 | 27 | 28 | 29 |
|---|---|---|---|---|---|---|---|---|---|---|---|---|---|---|---|---|---|---|---|
| Blood | 120 | 140 | 19 | 20 | 24 | 25 | 36 | 43 | 69 | 90 | 73 | 92 | 56 | 59 | 30 | 46 | 48 | 51 | 60 |
| Symptom | Healthy subject, | | | | | | Light | | | | Moderate | | | | Severe | | | | |
| | very light | | | | | | | | | | | | | | | | | | |
| Pathology | | | | | | | ∘ | ∘ | ∘ | ∘ | ∘ | ∘ | • | • | • | • | • | • | |
| Asthma | | | | | | | Light | Light | None | Light | None | None | None | Light | None | None | None | Light | None |
| S IgE | − | − | − | − | − | − | + | + | + | + | + | + | + | + | + | + | + | + | + |
| Eosinophil | B | B | B | B | B | A | B | C | C | C | C | C | B | C | C | C | C | B | C |
| T IgE | L | L | L | L | L | L | L | L | L | H | H | L | H | H | L | L | H | H | H |

(2) Differential Display Analysis:

A 3% dextran solution was added to whole blood drawn from a healthy subject and a patient, and this was left to stand at room temperature for 30 minutes to precipitate erythrocytes. The upper layer leukocyte fraction was collected, layered on top of Ficoll solution (Ficoll-Paque PLUS; Amersham Pharmacia Biotech), and centrifuged at 1500 rpm for 30 minutes at room temperature. The granulocyte fraction that collected in the lower layer was reacted with CD16 antibody magnetic beads at 4° C. for 30 minutes, and cells that had eluted without being trapped in the separation using MACS were used in the experiment as eosinophils.

Eosinophils prepared as described above were dissolved in Isogen (Nippon Gene; Wako Pure Chemicals), and from this solution, RNA was separated according to the protocol attached to Isogen. Chloroform was added, the mixture was stirred and centrifuged, and the aqueous layer was collected. Next, isopropanol was added, the mixture was stirred and centrifuged, and the precipitated total RNA was collected. DNase (Nippon Gene; Wako Pure Chemicals) was added to the collected total RNA, the mixture was reacted at 37° C. for 15 minutes, and RNA was collected by phenol-chloroform extraction followed by ethanol precipitation.

Fluorescent Differential Display (abbreviated to DD) analysis using total RNA thus prepared was carried out according to the literature (T. Ito et al., 1994, FEBS Lett. 351: 231-236). The total RNA was reverse transcribed to obtain cDNA. In the first BD-PCR, 0.2 μg each of total RNA was used for three types of anchor primers to synthesize cDNAs. In the second DD-PCR, 0.4 μg each of total RNA was used for the synthesis of cDNAs using three types of anchor primers. In both cases, the cDNAs were diluted to a final concentration equivalent to 0.4 ng/μl RNA and used for further experiments. The DD-PCR was carried out using an amount of cDNA equivalent to 1 ng RNA per reaction. The reaction mixture composition is shown in Table 2.

TABLE 2

| | | |
|---|---|---|
| cDNA (equivalent to 0.4 ng/μl RNA) | 2.5 | μl |
| Arbitrary primer (2 μM) | 2.5 | μl |
| 10x AmpliTaq PCR buffer | 1.0 | μl |
| 2.5 mM dNTP | 0.8 | μl |
| 50 μM anchor primer | 0.1 | μl |
| (GT15A, GT15C, or GT15G) | | |
| Gene Taq (5 U/μl) | 0.05 | μl |
| AmpliTaq (5 U/μl) | 0.05 | μl |
| dH₂O | 3.0 | μl |
| Total volume | 10.0 | μl |

The PCR was carried out at following condition: 1 cycle of “95° C. for 3 min, 40° C. for 5 min, and 72° C. for 5 min”; subsequently 30 cycles of “94° C. for 15 sec, 40° C. for 2 min, and 72° C. for 1 min”; after these cycles, 72° C. for 5 min; and then continuously 4° C.

Reactions were conducted using 287 primer pairs: i.e., anchor primers GT15A (SEQ ID NO: 2), GT15C (SEQ ID NO: 3), and GT15G (SEQ ID NO: 4) were used in combination with arbitrary primers AG 1 to AG 110, AG 111 to AG 199, and AG 200 to AG 287, respectively. As for the arbitrary primers, oligomers having 10 nucleotides with a GC content of 50% were designed and synthesized.

For gel electrophoresis, a 6% denaturing polyacrylamide gel was prepared, and 2.5 μl sample from the PCR was applied and run under 40 W for 210 min. After electrophoresis, the gel was scanned by Hitachi fluorescence imaging analyzer FMBIO II, and the gel image was obtained by detecting fluorescence.

Samples from both healthy subjects and patients were electrophoresed side-by-side, and the bands that showed variation in expression between each of the samples were isolated. Sequences were determined for the bands that were selected by visual judgment and indicated values of 0.1 or less in significance tests. Furthermore, sequences were determined for bands selected using an image analysis software, Bio-Image. Identical sequence clones in each of the bands were grouped, and were designated as consensus sequences. As a result, among the sequence determined bands, a band that can be uniquely defined as the “dominant sequence” was selected.

The selected consensus sequence was used as the query to perform a homology search through genembl and dbEST using BLAST in GCG. Herein, a sequence with 95% or more identity was determined as the sequence “with significant homology”.

As a result of such analysis, bands that showed increased expression specifically in patients were identified. The primer set used to amplify the identified “1835-17” band is shown in Table 3. The number in parenthesis after the sequence of the arbitrary primer is the SEQ ID NO. Furthermore, the nucleotide sequence of “1835-17” band is shown in SEQ ID NO: 1.

TABLE 3

| | | | Name of | Sequence of |
|---|---|---|---|---|
| | | Anchor | arbitrary | arbitrary primer |
| Band ID | bp | primer | primer | (SEQ ID NO) |
| 1835-17 | 365 | GT15A | AG00017 | CTTTGAGCGA (5) |

Furthermore, using the nucleotide sequence of the “1835-17” fragment (SEQ ID NO: 1) as the query, a BLAST homology search was performed through GenBank. As a result, the nucleotide sequence of SEQ ID NO: 1 mostly matched the nucleotide sequence of human intersectin 2 gene. Therefore, the previously identified nucleotide sequence of the “1835-17” fragment was confirmed to be a partial sequence of human intersectin 2 gene. Increase of expression of human intersectin 2 in eosinophils of allergic disease patient was a novel finding found by the present inventors. This finding reveals the usefulness of human intersectin 2 gene as an indicator gene for an allergic disease.

### EXAMPLE 2

### Quantification of Expression Level by ABI 7700

The expression of genes identified in Example 1 was analyzed by TaqMan method using ABI 7700. RNAs were prepared in the same manner as in Example 1 from 10 samples each of freshly collected eosinophils from healthy subjects and patients with light, moderate, and severe atopic dermatitis. The examination value profiles of healthy subjects and patients are shown in Table 4. Expression levels were quantified for the gene in the band identified in Example 1, and for β-actin gene, which is known to be an internal standard for correction.

TABLE 4

| | 1 | 2 | 3 | 4 | 5 | 6 | 7 | 8 | 9 | 10 |
|---|---|---|---|---|---|---|---|---|---|---|
| Blood | | | | | | | | | | |
| Symptom | None | | | | | | | | | |
| Pathology | None | | | | | | | | | |
| Asthma | None | | | | | | | | | |
| Mite IgG | − | | | | | | | | | |
| Eosinophil | B | B | B | A | B | B | B | A | A | B |
| Total | L | | | | | | | | | |
| IgE | | | | | | | | | | |
| | 11 | 12 | 13 | 14 | 15 | 16 | 17 | 18 | 19 | 20 |
| Blood | 80 | 109 | 125 | 130 | 131 | 164 | 170 | 197 | 205 | 215 |
| Symptom | Light | | | | | | | | | |
| Pathology | 602 | ∘ | ∘ | ∘ | ∘ | ∘ | ∘ | ∘ | ∘ | ∘ |
| Asthma | Light | None | None | Light | Light | Light | None | Light | None | Light |
| Mite IgG | + | + | + | + | + | + | + | + | + | + |
| Eosinophil | C | B | B | C | C | C | C | C | C | C |
| Total | L | L | H | H | H | L | L | L | L | L |
| IgE | | | | | | | | | | |
| | 21 | 22 | 23 | 24 | 25 | 26 | 27 | 28 | 29 | 30 |
| Blood | 101 | 147 | 162 | 179 | 196 | 210 | 218 | 219 | 226 | 232 |
| Symptom | Moderate | | | | | | | | | |
| Pathology | ∘ | ∘ | ∘ | • | • | • | • | ∘ | ∘ | ∘ |
| Asthma | None | None | None | Light | None | Light | None | None | None | None |
| Mite IgG | + | + | + | + | + | + | + | + | + | − |
| Eosinophil | B | C | C | B | A | C | C | B | C | A |
| Total | H | L | H | H | L | H | H | L | L | L |
| IgE | | | | | | | | | | |
| | 31 | 32 | 33 | 34 | 35 | 36 | 37 | 38 | 39 | 40 |
| Blood | 96 | 135 | 146 | 167 | 184 | 194 | 211 | 225 | 227 | 238 |
| Symptom | Severe | | | | | | | | | |
| Pathology | • | • | • | • | • | • | • | • | • | • |
| Asthma | None | None | None | Light | None | None | Light | None | None | None |
| Mite | + | + | + | + | + | + | + | + | + | + |
| IgG | | | | | | | | | | |
| Eosinophil | B | C | B | B | B | C | C | C | C | C |
| Total | L | H | H | L | L | H | H | H | H | H |
| IgE | | | | | | | | | | |

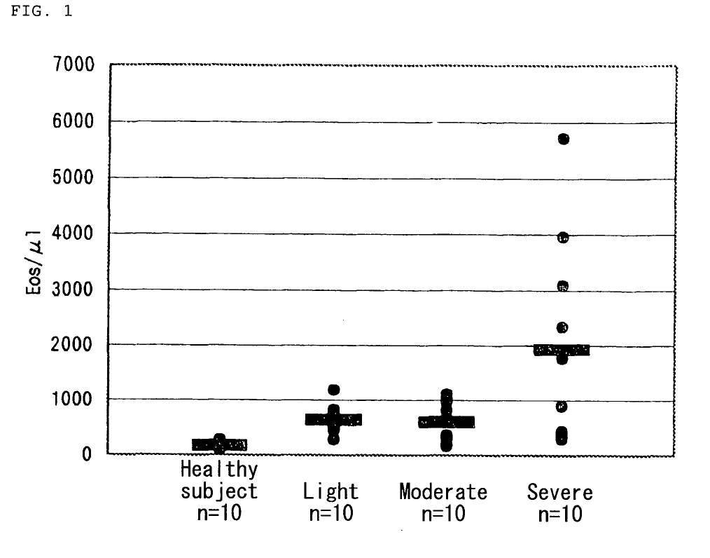

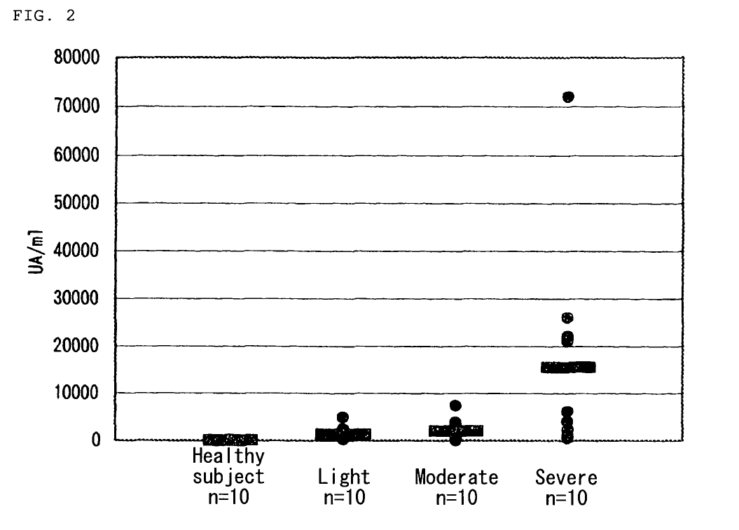

FIG. 1 (number of eosinophils) and FIG. 2 (total IgE) show plotted examination values of each group based on the examination value profiles of 10 samples each of healthy subjects and patients with light, moderate, and severe atopic dermatitis. The number of eosinophils in atopic dermatitis patients is evaluated as B to C, but when the actual measured values are compared, the measured values from severe patients alone are prominent, as apparent from FIG. 1. This shows that the number of eosinophils is difficult to utilize as an indicator for diagnosis of light or moderate atopic dermatitis.

A similar trend can be observed for the measured values of total IgE (FIG. 2). Specifically, marked increase in total IgE value is observed in severe patients, and a large difference compared to values in healthy subjects is not observed in light to moderate patients. Therefore, this shows that total IgE value is also difficult to use as an indicator for a light allergic disease.

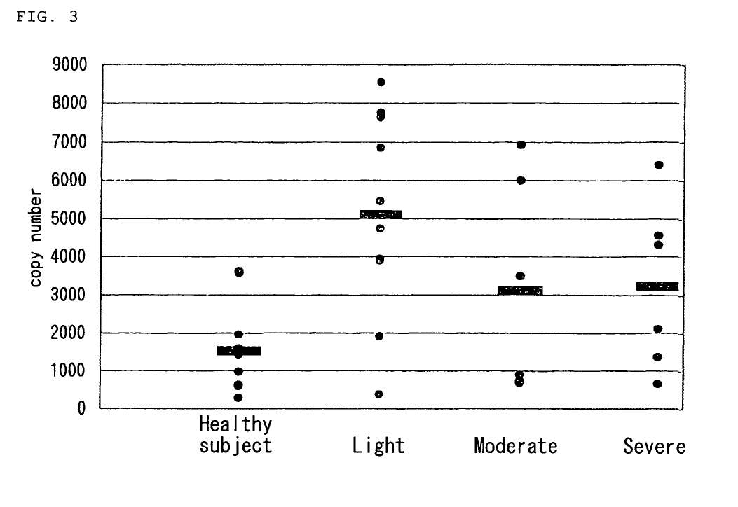

The primers and TaqMan probes used for measurements by ABI 7700 were designed by Primer Express (PE Biosystems) based on the sequence information for the gene. TaqMan probes are labeled on the 5′-end with FAM (6-carboxy-fluorescein), and on the 3′-end with TAMRA (6-carboxy-N,N,N′,N′-tetramethylrhodamine). The nucleotide sequences of the primers and the TaqMan probes used for the experiment are as shown in the SEQ ID NOs of Table 5. Primers and probes used for measuring β-actin were those included in TaqMan β-actin Control Reagents (PE Biosystems). The result of measurement is shown in FIG. 3. Furthermore, the average gene expression levels (AVERAGE: copy/ng (corrected value)) in clinical samples are summarized in Table 6.

TABLE 5

| ID | Forward | Reverse | Probe |
|---|---|---|---|
| 1835-17 | 6 | 7 | 8 |
| β-actin | 9 | 10 | 11 |

TABLE 6
Expression level of genes in clinical samples
(AVERAGE: copy/ng (corrected value))

| | Healthy | | | |
|---|---|---|---|---|
| Band ID | subjects | Light | Moderate | Severe |
| 1835-17 | 1520 | 5106 | 3107 | 3226 |

Using the above-mentioned data, parametric multiple comparison test and non-parametric multiple comparison test were carried out. Statistical analysis was carried out using SAS Pre-clinical Package of The SAS SYSTEM, Version 4.0 (SAS Institute Inc.). The results are shown in Table 7.

As apparent from Table 7, expression of the gene identified in the present invention was significantly increased due to atopic dermatitis (light). Therefore, this fact gives support to the diagnostic value of measuring the expression of these genes for atopic dermatitis.

TABLE 7

| | | | | |
|---|---|---|---|---|
| Band ID | Dunnet | P value | Tukey | P value |

Parametric multiple comparison

| | | | | |
|---|---|---|---|---|
| B1835-17 | Light > | 0.0037 | Light > | 0.0068 |
| | Normal | | Normal | |

Nonparametric multiple comparison

| | | | | |
|---|---|---|---|---|
| B1835-17 | Light > | 0.0071 | Light > | 0.0132 |
| | Normal | | Normal | |

### EXAMPLE 3

### Expression of the Indicator Gene in Various Blood Cells

Expression of each gene was examined in cells separated from peripheral blood collected from five normal healthy subjects. Separation of eosinophils (E) was carried out as described above. After the elution of eosinophils, neutrophils (N) were prepared by releasing the cells, which were trapped with CD16 antibody magnetic beads, from the magnetic field, eluting, and recovering. On the other hand, the monocyte fraction recovered in the middle layer by the Ficoll-centrifugation was separated into the fraction eluted from MACS CD3 antibody magnetic beads (mixture of M (monocyte) and B cell) and fraction trapped therein (T-cell fraction). Then, using MACS CD14 antibody magnetic beads, the eluted fraction was separated into the eluted fraction (B cell fraction) and trapped fraction (monocyte fraction), and those three fractions were referred to as the purified T cells, B cells, and monocytes.

Eosinophils were solubilized using Isogen, while neutrophils, T cells, B cells and monocytes were solubilized with RNeasy (Qiagen), and total RNA were extracted, treated with DNase (by the same methods as described above), and subjected to the gene expression analysis. Primers, probes, and others used were the same as above. Average expression levels (AVERAGE: copy/ng (corrected value)) in these blood cells are shown in Table 8.

TABLE 8
Expression level of genes in various blood cells
(AVERAGE: copy/ng (corrected value))

| Band ID | Eosinophil | Neutrophil | B cell | T cell | Monocyte |
|---|---|---|---|---|---|
| 1835-17 | 6813 | 6573 | 1621 | 1427 | 693 |

### EXAMPLE 4

### Change in Gene Expression in Human Peripheral Blood Eosinophils Due to Stimulation by Cytokines

Since eosinophils are considered to be the central inflammatory cells in allergic inflammation, the present inventors examined effects of cytokines on gene expression relating to growth, differentiation, migration and accumulation to a local region, and activation of eosinophils.

The change in gene expression due to cytokine stimulation in eosinophils isolated from 100 ml of peripheral blood from a healthy subject was studied. Isolation of eosinophils was carried out as described above. Eosinophils were plated onto a 24-well plate (1×10⁶ cells/mL). The plate was pre-coated with 1% BSA (immobilizing blocking buffer) at room temperature for 2 hours in order to prevent activation due to adhesion of eosinophils. As cytokines, 0.1, 1, and 10 ng/ml each of interleukin 5 (IL-5), interleukin 4 (IL-4), interferon γ (IFNγ), granulocyte macrophage colony stimulating factor (GM-CSF), and eotaxin were added to each well, and this was cultured for 3 hours in DMEM supplemented with 10% FCS. All of these cytokines are those considered to be related to activation of eosinophils and onset of allergies.

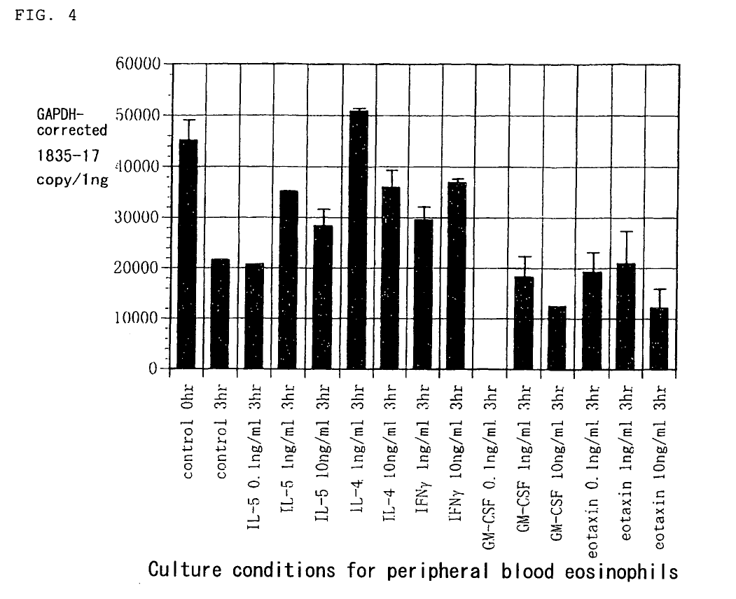

RNAs were prepared in the same manner as in Example 1 for each of the treated eosinophils, and were subjected to gene expression analysis. The primers, probes, and such used were the same as described above. The result at 3 hours after starting the culture is shown in FIG. 4 (value was corrected with GAPDH for the number of copies per 1 ng of RNA).

Among the cytokines used in the experiment, IL-5 extends the life-time of eosinophils by activating the eosinophils. Therefore, IL-5 treatment increases the expression levels of anti-apoptotic genes, bcl-2 and bax, in eosinophils. Since expression of “1835-17” (intersectin 2 gene) increases similarly, as indicated in FIG. 4, their expression may be correlated to extension of the life-time of eosinophils, and their relationship to induction and exacerbation of the pathology of allergies was suggested.

Furthermore, the expression of “1835-17” (intersectin 2 gene) was also induced by IFNγ and IL-4. Regarding these cytokines, there are not many findings relating to gene expression in eosinophils. However, since all are important factors for the onset of allergies, induction of expression of the genes in eosinophils by these cytokines may suggest the possibility that “1835-17” (intersectin 2 gene) is related to the pathology and exacerbation of allergic diseases.

### EXAMPLE 5

Using a 2,4-dinitrofluorobenzene (DNFB) coated mouse as a model for an allergic reaction, changes in the expression level of the intersectin 2 gene in the mouse were observed. DNFB is a substance that is used as a hapten to cause allergic dermatitis reaction in a laboratory animal such as a mouse.

A sensitizing solution having the following composition was prepared.

Acetone: Olive oil=4:1 (=2 mL: 500 μL)

DNFB 0.4% (=10 μL)

The challenge solution had the following composition.

Acetone: Olive oil=4:1 (=2 mL: 500 μL)

DNFB 0.2% (=5 μL)

The administration schedule is as follows.

Day 0: The abdominal region was shaved by an electric clipper and 25 μL of the sensitizing solution was applied to the abdominal region.

Day 1: In a similar manner to the previous day, 25 μL of the sensitizing solution was applied to the abdominal region.

Day 5: 5 μL/ear of challenge solution was applied to both ears.

Day 6: Thicking of the ears is confirmed, and then RNAs are extracted following the protocol of TRIZOL Reagent.

A mouse to which the solvent alone without DNFB had been coated (“CONT. mouse”) was prepared as the control. Furthermore, expression of the intersectin 2 gene was observed in a mouse to which steroid was orally administered prior to DNFB application (DS mouse) More specifically, when applying DNFB, steroid was orally administered every day from day 0 to day 6, 30 minutes before the application. As the steroidal agent, prednisolone adjusted to 1 mg/mL with methyl cellulose was orally administered at 0.1 mL/10 g body weight of the mouse. Simultaneously, expression of the intersectin 2 gene was observed in a mouse to which DNFB was not applied and steroid (prednisolone) alone was orally administered (“S mouse”).

The expression level of the intersectin 2 gene was quantified by the TaqMan method (as mentioned above) using ABI 7700 and using mRNA sample extracted from the mouse ear tissue. The nucleotide sequences of the primers and probe used for the TaqMan method are shown below. The primers and probe used in this experiment were designed based on the nucleotide sequence (GenBank Accession No. AF132480)) predicted to encode the mouse intersectin 2 gene (EMBO J. 18 (5), 1159-1171 (1999)).

Primer

SEQ ID NO: 12

1835-1F: 5-ACCAGCAAGAGTTCTCTATAGCTATG-3/

SEQ ID NO: 13

1835-1R: 5-CTGTAAGATGATGCATGAGGCAATGT-3/
TaqMan probe

SEQ ID NO: 14

1835-17: 5-famTCATCAGCCATTGCCTCCAGTTGCACCtamura-3/

PCR was carried out using 25 μL of 2× Master Mix without UNG, 1.25 μL of 40×MultiScribe and RNase Inhibitor Mix, 0.25 μL of 10 μM primer, and 0.625 μL of 4 μM TaqMan Probe derived from One-step RT-PCR Master Mix Reagents (PE Biosystems), and RNA (2 ng)+DEPC resulting in total volume of 50 μL. It was performed under conditions of 94° C. for 5 min, and 40 cycles of 94° C. for 30 sec, 55° C. for 30 sec, and 72° C. for 1 min. For creating the calibration curve, serial five-fold dilutions of DNA (50 ng, 10 ng, 2 ng, 0.4 ng, and 0.08 ng) were prepared. To correct the differences of cDNA concentration among the samples, a similar quantitative analysis was performed on the 18S gene, and the value for the gene of interest was divided by the value for 18S, and indicated as taking the value of CONT. as 1.

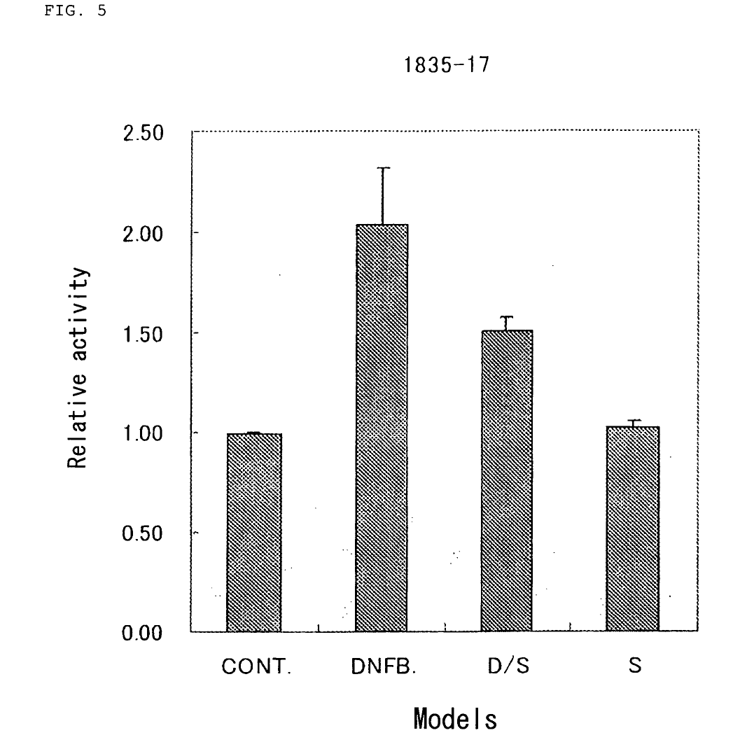

Four each of control mice (CONT.), DNFB applied mice (DNFB.), DNFB applied mice to which steroid was administered (D/S), and steroid administered mice (S) were used, and the obtained results were analyzed by t test (two-sample test assuming equal variance). The result is shown in Table 9. Furthermore, the result is shown in FIG. 5.

TABLE 9

| | Average | S.E.M. |
|---|---|---|
| CONT. | 1.00 | 0.00 |
| DNFB | 2.04 | 0.28 |
| D/S | 1.51 | 0.07 |
| S | 1.04 | 0.05 |

Significant differences were found at p<0.05 between CONT., which is the control, and the DNFB applied group, and between the control and the DNFB applied group to which steroid was administered (D/S). The mouse intersectin 2 gene level was confirmed to increase in an inflammatory allergy model. This supports the close relationship between intersectin 2 gene and allergic reaction. Particularly, significant increase of the expression of the intersectin 2 gene even in the steroid administered model strongly suggests that this gene is related to the cause of allergic reaction. More specifically, the intersectin 2 gene is not induced as a result of an allergic reaction. Therefore, the intersectin 2 gene is useful as the target molecule for treatment and prevention of allergic diseases.

### INDUSTRIAL APPLICABILITY

The present invention provides genes that show increased expression in eosinophils of patients with early stage atopic dermatitis. Genes that show elevated expression prior to increase of eosinophils can be utilized as highly sensitive indicators for allergic symptoms. Diagnosis of allergic symptoms at a stage when increase of eosinophils is not observable is normally difficult. However, the indicators provided by the present invention enable early diagnosis that had been difficult with the diagnosis indicators to date. Enabled early diagnosis makes it possible to select accurate therapeutic methods even for early stage allergic diseases.

Increase of eosinophils is an important step in allergic reactions. Thus, genes that show increased expression in eosinophils prior to disease-induced increase in eosinophils are considered to play an important role, especially in the early stages of allergic diseases. Therefore, suppressing the expression and activity of the indicator gene of this invention becomes a strategic target for therapy of allergic diseases, and such genes can be expected to be useful as novel clinical diagnostic indicators for monitoring in such novel therapeutic methods.

Expression levels of indicator genes provided by the present invention can be easily detected regardless of the types of allergens. Therefore, pathological conditions of allergic diseases can be comprehensively understood.

In addition, using peripheral blood eosinophils as a specimen, the expression level of genes can be analyzed in a much less invasive manner to patients according to the method for testing for allergic diseases of the present invention. Furthermore, according to the gene expression analysis method of the present invention, in contrast to protein measurements such as ECP, highly sensitive measurement with a trace sample can be accomplished. Gene analysis technique trends toward high-throughput and lower prices. Therefore, the test method according to the present invention is expected to become an important bedside diagnostic method in the near future. In this sense, these genes associated with pathological conditions are highly valuable in diagnosis.

1. A method of testing for an allergic disease, said method comprising the steps of:
a) measuring the expression level of an indicator gene in a biological sample of a test subject; and
b) comparing the expression level of the indicator gene in the biological sample of a test subject to that of a healthy subject,
wherein the indicator gene is intersectin 2 gene or a gene functionally equivalent thereto.

2. The testing method of claim 1, wherein the allergic disease is atopic dermatitis.

3. The testing method of claim 1, wherein the expression level of the gene is measured by cDNA PCR.

4. The testing method of claim 1, wherein the expression level of the gene is measured by detecting a protein encoded by the gene.

5. The method of claim 1, wherein the biological sample contains peripheral blood eosinophil cells.

6. A reagent for testing for the presence of an allergic disease, which comprises an oligonucleotide containing at least 15 nucleotides of a nucleotide sequence complementary to a polynucleotide containing the nucleotide sequence of intersectin 2 gene or a gene functionally equivalent thereto or to a complementary strand of the polynucleotide.

7. A reagent for testing for an allergic disease, which comprises an antibody that recognizes a peptide containing an amino acid sequence encoded by intersectin 2 gene or by a gene that is functionally equivalent thereto.

8. A method of screening for a therapeutic agent for an allergic disease, said method comprising the steps of:
(1) contacting a candidate compound with cells expressing an indicator gene;
(2) measuring the expression level of the indicator gene; and
(3) selecting a compound that decreases the expression level of the indicator gene compared to a control,
wherein the indicator gene is intersectin 2 gene or a gene functionally equivalent thereto.

9. The method of claim 8 wherein the cells are eosinophil cells.

10. The method of screening for a therapeutic agent for an allergic disease, said method comprising the steps of:
(1) administering a candidate compound to a test animal;
(2) measuring the expression intensity of an indicator gene in a physiological sample of the test animal; and
(3) selecting a compound that decreases the expression level of the indicator gene compared to a control,
wherein the indicator gene is intersectin 2 gene or a gene functionally equivalent thereto.

11. A method of screening for a therapeutic agent for an allergic disease, said method comprising the steps of:
(1) contacting a candidate compound with a cell transfected with a vector comprising a transcription regulatory region of an indicator gene and a reporter gene that is expressed under the control of the transcription regulatory region;
(2) measuring the activity of the reporter gene; and
(3) selecting a compound that decreases the expression level of the reporter gene compared to a control,
wherein the indicator gene is intersectin 2 gene or a gene functionally equivalent thereto.

12. A method of screening for a therapeutic agent for an allergic disease, said method comprising the steps of:
(1) contacting a candidate compound with a protein encoded by an indicator gene;
(2) measuring the activity of the protein; and
(3) selecting a compound that decreases the activity of the protein, compared to a control,
wherein the indicator gene is intersectin 2 gene or a gene functionally equivalent thereto.

13. A therapeutic agent for an allergic disease, which comprises as an active ingredient a compound obtainable by the screening method of any one of claims 8, 10, 11, and 12.

14. A therapeutic agent for an allergic disease, which comprises an antisense DNA of an indicator gene or a portion thereof as the main ingredient, wherein the indicator gene is intersectin 2 gene or a gene functionally equivalent thereto.

15. A therapeutic agent for an allergic disease, which comprises as the main ingredient an antibody that binds to a protein encoded by an indicator gene, wherein the indicator gene is intersectin 2 gene or a gene functionally equivalent thereto.

16. Use of a transgenic non-human vertebrate as an animal model for an allergic disease, which has increased expression intensity of an indicator gene in eosinophil cells, wherein the indicator gene is intersectin 2 gene or a gene functionally equivalent thereto.

17. A kit for screening for a therapeutic agent for an allergic disease, which comprises an oligonucleotide containing at least 15 nucleotides of a nucleotide sequence complementary to a polynucleotide containing the nucleotide sequence of an indicator gene or to a complementary strand of the polynucleotide, and a cell that expresses the indicator gene, wherein the indicator gene is intersectin 2 gene or a gene functionally equivalent thereto.

18. A kit for screening for a therapeutic agent for an allergic disease, which comprises an antibody that recognizes a peptide comprising the amino acid sequence encoded by an indicator gene, and a cell that expresses the indicator gene, wherein the indicator gene is intersectin 2 gene or a gene functionally equivalent thereto.
